(19) United States
(12) Patent Application Publication
(10) Pub. No.: US 2010/0184864 A1
(43) Pub. Date: Jul. 22, 2010

(54) Composite Material and Method for Production Thereof

(75) Inventors: Tadashi Hamajima, Higashi-Osaka-shi (JP); Tomomi Yanagibayashi, Higashi-Osaka-shi (JP); Ryuji Arakawa, Higashi-Osaka-shi (JP); Morihiro Aoyagi, Higashi-Osaka-shi (JP); Masayoshi Kushi, Higashi-Osaka-shi (JP)

Correspondence Address: MARSHALL, GERSTEIN & BORUN LLP, 233 SOUTH WACKER DRIVE, 6300 WILLIS TOWER, CHICAGO, IL, 60606-6357, US

(73) Assignee: HOUSE FOODS CORPORATION, Higashi-Osaka-shi, Osaka (JP)

(21) Appl. No.: 12/665,021

(22) PCT Filed: Jun. 27, 2008
(86) PCT No.: PCT/JP2008/061744

§ 371 Date: Dec. 16, 2009

(30) Foreign Application Priority Data
Jun. 29, 2007 (JP) 2007-172987

(51) Int. Cl. A61K 31/16; A61K 8/42; A61P 1/00; A61P 3/00; A61P 9/00; A23D 9/007

(52) U.S. Cl. 514/627; 426/611

(57) ABSTRACT

Disclosed are: a material which contains a lipophilic component having a pungent taste or flavor such as a spicy component (e.g., a capsaicin) and a bitter component (e.g., a turmeric extract), in which the pungent taste and/or flavor of the lipophilic component can be reduced effectively, and in which the separation among components contained therein is not caused; a method for producing the material; a composition such as a food, a beverage, a cosmetic and a medicine, which contains the material; and a liquid composition having such a form that the material is dispersed in water. Specifically disclosed are: a composite material comprising a lipophilic component having a pungent taste and/or flavor, a plant-derived cholesterol ester and a cyclodextrin; a composition comprising the composite material; a liquid composition which comprises the composite material, water and a thickening agent and has such a form that the composite material is dispersed in water; and a method for producing a composite material comprising a lipophilic component having a pungent taste and/or flavor, a plant-derived cholesterol ester and a cyclodextrin, which comprises mixing the lipophilic component, the plant-derived cholesterol ester and the cyclodextrin in the presence of water to form the composite material.

### TECHNICAL FIELD

The present invention relates to: a composite material containing a lipophilic component having an irritating taste or odor typified by pungent components such as capsaicins and bitter components such as a turmeric extract; and a production method thereof.

### BACKGROUND ART

Capsaicins, which are pungent components of capsicum pepper, for example, and one of the lipophilic components having an irritating taste or odor, are known to have various effects useful for organisms, such as an effect of appetite stimulation, an effect of vasodilation and vasoconstriction, an effect of increasing salivation, an effect of increasing gastric acid secretion, an effect of increasing peristaltic movement of the intestinal tract, an effect of decreasing the cholesterol level in the circulatory system, an effect of increasing energy metabolism, and an effect of increasing the release of bioactive peptides. However, capsaicins have a strong pungency, and hence the application range thereof to foods and beverages has been limited.

To inhibit the pungency of capsaicins, novel glycoside capsaicinoids have been proposed which are obtained by modifying the molecular structure of capsaicinoids to eliminate their strong pungency (Patent Document 1). However, the glycoside capsaicinoids are novel chemically synthesized compounds, and hence are not approved for use in foods or beverages.

Moreover, a masking agent which is characterized by including a polyglycerin condensed ricinoleate and a food including the masking agent have been proposed (Patent Document 2). Specifically, a masking agent-containing chili oil has been disclosed which is obtained by adding 0.1% of a capsicum pepper extract oil and 0.5% of hexaglycerin condensed ricinoleate to sesame oil. However, the application range of this masking agent is limited to foods and beverages containing a large oil component, and in addition, this masking agent is likely to affect the flavor of foods and beverages because of a waxy odor thereof.

Furthermore, an edible microcapsule has been proposed which is characterized in that an edible fat and oil containing capsaicin is used as a core material, a wall membrane is formed of protein and a coacervate agent, and a trans glutaminase is used as an agent for curing and crosslinking the wall membrane (Patent Document 3). However, the present inventors have actually prepared this edible microcapsule and stirred this edible microcapsule with hot water (at 97° C.) in a beaker, and as a result, the core material leaked and floating oil was observed. Therefore, the edible microcapsule was found not to be suitable for beverages.

Patent Document 1: Japanese Patent No. 3156240

Patent Document 2: Japanese Patent Application Publication No. 2002-65177

Patent Document 3: Japanese Patent Application Publication No. 2003-47432

### DISCLOSURE OF THE INVENTION

An object of the present invention is to provide: a material including lipophilic components having an irritating taste or odor typified by pungent components such as capsaicins and bitter components such as a turmeric extract, the material being capable of effectively inhibiting the irritating taste and/or odor of the lipophilic components and of avoiding the separation of the components; and a production method thereof.

Another object of the present invention is to provide a composition, such as foods, beverages, cosmetics and pharmaceutical drugs, including the above-described material blended therein.

Moreover, still another object of the present invention is to provide a liquid composition in which the above-described material is in a water dispersed form.

The present invention provides a composite material comprising: a lipophilic component having an irritating taste and/or odor; a phytosterol ester; and a cyclodextrin.

In addition, the present invention provides a composition comprising the composite material blended therein.

Further, the present invention provides a liquid composition comprising the composite material, water and a thickener, wherein the composite material is in a water dispersed form.

Furthermore, the present invention provides a method for producing a composite material comprising: a lipophilic component having an irritating taste and/or odor; a phytosterol ester; and a cyclodextrin, the method comprising the step of mixing the lipophilic component, the phytosterol ester and the cyclodextrin with each other in the presence of water, to thereby form the composite material.

The present invention makes it possible to provide: a material including lipophilic components having an irritating taste or odor typified by pungent components such as capsaicins and bitter components such as a turmeric extract, the material being capable of effectively inhibiting the irritating taste and/or odor of the lipophilic components and of avoiding the separation of the components; and a production method thereof.

The present invention also makes it possible to provide a composition, such as foods, beverages, cosmetics and pharmaceutical drugs, including the above-described material blended therein.

Moreover, the present invention makes it possible to provide a liquid composition in which the above-described material is in a water dispersed form.

### BEST MODES FOR CARRYING OUT THE INVENTION

A composite material of the present invention by which an irritating taste and/or odor from a lipophilic component having the irritating taste and/or odor is inhibited includes: the lipophilic component having the irritating taste and/or odor; a phytosterol ester; and a cyclodextrin.

Typical examples of the lipophilic component having an irritating taste and/or odor and used in the present invention include capsaicins, which are one of the lipophilic pungent components. The capsaicins include capsaicin, dihydrocapsaicin, nordihydrocapsaicin, homocapsaicin, vanillylnonanamide, and vanillyl butyl ether. Since they contain a large amount of capsaicins, capsicum pepper extracts such as capsicum oleoresins are suitably used as a raw material containing capsaicins.

Meanwhile, examples of the lipophilic component other than the capsaicins include: (6)-gingerol, (6)-shogaol, zingerone, and (8),(10)-shogaol, which are pungent components of ginger; piperine and piperanine, which are pungent components of pepper; sanshool, which is a pungent component Japanese pepper; and allyl isothiocyanate, which is a pungent component of Japanese horseradish, Western horseradish, mustard and the like. A pepper extract, a ginger extract, and a Japanese pepper extract can be suitably used as a raw material containing pungent components of pepper, ginger, and Japanese pepper, respectively.

Besides the pungent components, the present invention can be applied to lipophilic bitter components such as a turmeric extract containing a lipophilic component with a bitter taste. It was found that the present invention reduces not only irritating tastes, but also irritating odors of the above-mentioned Japanese pepper extract, allyl mustard oil, and the like.

The phytosterol ester used in the present invention is a substance obtained by ester-bonding a fatty acid to a hydroxyl group in the sterol skeleton of a plant sterol. Examples of a production method of the phytosterol ester include: an enzymatic method utilizing an enzyme; and the like. Examples of the enzymatic method include: a method of obtaining the phytosterol ester by mixing the phytosterol and the fatty acid and by causing reaction therebetween (at 30 to 50° C. for approximately 48 hours), with a lipase or the like used as a catalyst; and the like. Other synthesis methods include: a method of obtaining the phytosterol ester by esterification which involves dehydration of a plant sterol produced from soybean or the like with a fatty acid obtained from rapeseed oil, corn oil, or the like, in the presence of a catalyst; and the like.

Examples of the plant sterol include sterols contained in vegetable fats and oils, and the like. For example, the plant sterol may be one extracted and purified from a vegetable fat or oil of soybean, rapeseed, cottonseed oil, or the like. The plant sterol may be a mixture containing β-sitosterol, campesterol, stigmasterol, brassicasterol, fucosterol, dimethylsterol, and the like. For example, a soybean sterol contains 53 to 56% of sitosterol, 20 to 23% of campesterol and 17 to 21% of stigmasterol. As the plant sterol, one which is commercially-available as “phytosterol F” (produced by TAMA BIOCHEMICAL CO., LTD.) can also be used.

The fatty acid may be plant-derived, for example, derived from rapeseed oil or palm oil, or animal-derived. Examples of the fatty acid include myristic acid, stearic acid, palmitic acid, arachidonic acid, oleic acid, linoleic acid, α-linolenic acid, γ-linolenic acid, eicosapentaenoic acid, docosahexaenoic acid, palmitoleic acid, lauric acid, and the like.

Preferable examples of the phytosterol ester include: phytosterol esters each obtained from a phytosterol derived from soybean and a fatty acid derived from rapeseed oil; phytosterol esters each obtained from a phytosterol derived from soybean or rapeseed and a fatty acid derived from palm oil; and the like. The former includes “San Sterol NO. 3” of San-Ei Gen F. F. I., Inc., and the latter includes “phytosterol fatty acid ester” of TAMA BIOCHEMICAL CO., LTD.

The cyclodextrin used in the present invention refers to a cyclic non-reducing maltooligosaccharide, whose constitutional unit is glucose. Even though any one of α-cyclodextrin with six glucose units, β-cyclodextrin with seven glucose units, and γ-cyclodextrin with eight glucose units may be used, γ-cyclodextrin is preferable since γ-cyclodextrin is decomposed by human digestive enzymes and since γ-cyclodextrin is easy to use for foods and beverages, particularly for beverages because of its high solubility in water.

The composite material of the present invention can be obtained by mixing the lipophilic component, the phytosterol ester, and the cyclodextrin in the presence of water. The amount of the phytosterol ester which is present in producing the composite material of the present invention varies depending on the target lipophilic component; when the lipophilic component is, for example, capsaicins, the amount of the phytosterol ester is preferably 30 to 30000 parts by weight, relative to 1 part by weight of capsaicins. Meanwhile, the amount of the cyclodextrin is, for example, preferably 0.01 to 1000 parts by weight, and more preferably 0.1 to 100 parts by weight, relative to 1 part by weight of the phytosterol ester. The amount of water which is present in producing the composite material is, for example, preferably 0.01 to 100 parts by weight, and more preferably 0.1 to 10 parts by weight, relative to 1 part by weight of the cyclodextrin. Meanwhile, in producing the composite material of the present invention, the mixing is performed with warming to preferably 40 to 90° C., and more preferably 50 to 85° C.

More specifically, the composite material of the present invention can be produced by any one of the following methods (1) to (3); however, in order to more effectively inhibit the taste or odor of the lipophilic component, the method (1) is particularly preferable.

(1) dissolving the lipophilic component in the phytosterol ester; preparing a mixture containing the cyclodextrin and water; and mixing the phytosterol ester in which the lipophilic component is dissolved with the mixture.
(2) preparing a mixture containing the cyclodextrin, water and the phytosterol ester; and mixing the lipophilic component and water with the mixture.
(3) preparing a mixture containing the lipophilic component and the cyclodextrin; and mixing water and the phytosterol ester with the mixture.

Of these methods, the method (1) will be more specifically described below. When the lipophilic component is, for example, capsaicins, 1 part by weight of capsaicins is dissolved in 30 to 30000 parts by weight of the phytosterol ester, in the step of dissolving the lipophilic component in the phytosterol ester, although this varies depending on the target lipophilic component. For dissolving the lipophilic component in the phytosterol ester, the lipophilic component is preferably dissolved in a way that the lipophilic component is added to the phytosterol ester, and the resultant is warmed to 40 to 80° C., preferably to 50 to 70° C. Alternatively, the lipophilic component may be dissolved in a way that the phytosterol ester is warmed to 40 to 80° C., preferably 50 to 70° C., in advance, and the lipophilic component is added thereto.

In the step of preparing the mixture containing the cyclodextrin and water, the amounts of the cyclodextrin and water are not particularly limited, as long as the composite material can be formed later with these amounts. For example, the amount of the cyclodextrin is, for example, 0.01 to 1000 parts by weight and preferably 0.1 to 100 parts by weight relative to 1 part by weight of the phytosterol ester. The amount of water is, for example, 0.01 to 100 parts by weight, and preferably 0.1 to 10 parts by weight, relative to 1 part by weight of the cyclodextrin.

In the step of mixing the phytosterol ester in which the lipophilic component is dissolved with the above-described mixture, the mixing is continued until the composite material of the present invention is formed. The thus formed composite material is deposited in the form of grains in the lower part of water after the mixing is stopped and the mixture is allowed to stand for a while. Note that the mixing here is performed preferably by using a mixing apparatus with high shearing force, such as a kneader, for forming the composite material by thoroughly kneading these components.

The obtained composite material can be in any form. For example, by using a vehicle or the like, the composite material may be formed into a powder form or a granular form. The composite material may be in a liquid form or paste form where the composite material is dispersed or emulsified in a solvent such as water.

The thus obtained composite material of the present invention is advantageous in that the irritating taste and/or odor of the lipophilic component is effectively inhibited. The inhibition of the irritating taste and/or odor in the composite material of the present invention has a mechanism different form that of what is called masking involving addition of a sweet component, or the like. It is uncertain what structure the composite material of the present invention has; however, it is conceivable that at least the lipophilic component contained in the composite material of the present invention is in such a state that the lipophilic component cannot bind to the receptor of the taste.

In addition, the composite material of the present invention is advantageous in that the oil components do not separate. Incidentally, when a composite material is produced by using a different oil instead of the phytosterol ester, a homogeneous composite material cannot be obtained and the oil component separates. Hence, when blended into a beverage or the like, the composite material has a problem that the oil components float and attaches to the inner surface of a container. In contrast, the composite material of the present invention is advantageous in that a homogeneous composite material can be obtained, and that the oil components do not separate, and hence the oil components do not attach onto the inner surface of a container, when blended into a beverage or the like, with the use of the phytosterol ester.

Moreover, the composite material of the present invention is also thermally stable. For example, even when the composite material is blended into a food or beverage, and heated to 65 to 100° C., the irritating taste and/or odor of the lipophilic component can be inhibited, and no oil components separate.

The composite material of the present invention is easily dispersed in water. Hence, the composite material can be blended into foods or beverages, pharmaceutical drugs, cosmetics and the like, and provided as various compositions.

More specifically, examples of the food or beverage into which the composite material of the present invention is blended include beverages, jellies, tablets, and the like. Here, a case where the composite material of the present invention is blended into a beverage is taken as an example. A heat-sterilized beverage in a container can be produced, for example, as follows. The composite material of the present invention is added to water. An acidulant is added thereto to adjust the pH to 4.0 or less, preferably 2.5 to 3.5. Raw materials such as a sweetener, a fruit juice, a flavor, a coloring agent, and vitamin C are added and mixed thereto. This mixture is heated to 65 to 100° C. for sterilization treatment. Then this mixture is filled into a container, and sealed. Moreover, a jelly in a container can be produced by adding a gelatinizing agent to the above-mentioned raw materials.

The composition of the present invention comprising the composite material, water and a thickener can be provided as a liquid composition in which the composite material is in a water dispersed form. Specifically, although the composite material tends to be deposited in water, the inclusion of the thickener makes it possible to provide a liquid composition in which the composite material is dispersedly held in water. This liquid composition can also be provided as a liquid composition in a container, such as a beverage in a container. In this case, there are advantages in that the oil components do not separate in the container and hence the oil components do not attach onto the inner surface of the container.

Here, examples of the thickener include gellan gum, fermentation-derived cellulose, xanthan gum, gum arabic, tamarind gum, guar gum, locust bean gum, karaya gum, tara gum, agar, gelatin, pectin, soybean polysaccharides, CMC (carboxymethylcellulose), carrageenan, microcrystalline cellulose, propylene glycol alginate, and the like. Of these, fermentation-derived cellulose is preferably used, from the viewpoint that the composite material is dispersed uniformly in water and the composite material has a favorable texture when ingested orally.

The amount of the thickener is not particularly limited, as long as the composite material can be dispersed in water with the amount. For example, it is preferable that 0.01 to 1.0% by weight of the thickener be contained in the liquid composition.

### INDUSTRIAL APPLICABILITY

The composite material of the present invention including the lipophilic component having an irritating taste and/or odor; the phytosterol ester; and the cyclodextrin is a composite material where the irritating taste and/or odor of the lipophilic component is inhibited. Hence, the composite material of the present invention can be applied to foods, beverages, cosmetics, pharmaceutical drugs, and the like, to which a lipophilic component having an irritating taste and/or odor has not been applicable so far. Therefore this makes it possible to provide foods, beverages, cosmetics, pharmaceutical drugs, and the like, having various effects useful for organisms such as an effect of appetite stimulation, an effect of vasodilation•vasoconstriction, an effect of increasing salivation, an effect of increasing gastric acid secretion, an effect of increasing peristaltic movement of the intestinal tract, an effect of decreasing the cholesterol level in the circulatory system, an effect of increasing energy metabolism, and an effect of increasing the release of bioactive peptides.

### EXAMPLES

### Example 1

### (1) Production of Composite Material

To 0.303 parts by weight of a phytosterol ester, 0.024 parts by weight of a capsicum oleoresin were added. With stirring, the mixture was warmed to 60° C., and the capsicum oleoresin was dissolved. Thus a phytosterol ester in which capsaicins were dissolved was prepared.

Separately, 8.15 parts by weight of γ-cyclodextrin and 4.075 parts by weight of water (at 60° C.) were mixed with each other, to thereby prepare a mixture (a paste).

To the mixed paste, the phytosterol ester in which the capsaicins were dissolved was added. The mixture was kneaded for 10 minutes by using a mortar, while being warmed at 60° C. Thus, a composite material was produced.

### (2) Production of Beverage

To the composite material, 87.338 parts by weight of water was added to be mixed therewith. Subsequently, 0.1 parts by weight of citric acid and 0.01 parts by weight of trisodium citrate were added to be mixed therewith. Next, this mixture was warmed to 93° C. with stirring, and this temperature was kept for 3 minutes to perform heat sterilization. Thereafter, this mixture was filled into a container, and cooled. Thus, a beverage in the container was produced. Note that the beverage had a pH of 3.1. The thus obtained beverage had almost no perceptible pungency. Note that the following table 1 shows the composition of the beverage.

TABLE 1

| Composition of Beverage | Parts by weight |
|---|---|
| Capsicum oleoresin (content of capsaicins: 4.25% by | 0.024 |
| weight) | |
| phytosterol ester | 0.303 |
| (San-Ei Gen F.F.I., Inc., “San Sterol NO. 3”) | |
| γ-cyclodextrin | 8.15 |
| (Cyclochem Co., Ltd., “CAVAMAX W8 Food”) | |
| Water | 91.413 |
| Citric acid (San-Ei Gen F.F.I., Inc.) | 0.1 |
| Trisodium citrate (San-Ei Gen F.F.I., Inc.) | 0.01 |

### Example 2

### (1) Production of Composite Material

To 0.185 parts by weight of a phytosterol ester (San-El Gen F. F. I., Inc. “San Sterol NO. 3”), 0.015 parts by weight of a capsicum oleoresin were added. With stirring, the mixture was warmed to 60° C., and the capsicum oleoresin was dissolved. Thus, a phytosterol ester in which capsaicins were dissolved was prepared.

Separately, 5.00 parts by weight of γ-cyclodextrin and 2.5 parts by weight of water (at 60° C.) were mixed with each other, to thereby prepare a mixture (a paste).

To the mixed paste, the phytosterol ester in which the capsaicins were dissolved was added. The mixture was kneaded for 10 minutes by using a mortar, while being warmed to 60° C. Thus a composite material was produced.

### (2) Production of Beverage

To the composite material, 92.19 parts by weight of water were added to be mixed therewith. Subsequently, 0.1 parts by weight of citric acid and 0.01 parts by weight of trisodium citrate was added to be mixed therewith. Next, this mixture was warmed to 93° C. with stirring, and this temperature was kept for 3 minutes to perform heat sterilization. Thereafter, the mixture was filled into a container, and cooled. Thus, a beverage in the container was produced. Note that this beverage had a pH of 3.1.

The thus obtained beverage had almost no perceptible pungency. Note that the following Table 2 shows the composition of the beverage.

### Comparative Example 1

In accordance with the description in Patent Document 3, an edible microcapsule including, as the core material, fat and fatty oil containing approximately 0.15% by weight of a capsicum pepper extract was prepared. Into a beaker with a capacity of 200 ml, 5 parts by weight of this edible microcapsule and 95 parts by weight of hot water (water temperature: 97° C.) were added, and stirred manually with a spatula. As a result, oil floated on the water surface, and also a viscous substance was deposited, thereby giving a whitish liquid. Thus, said liquid was unstable for use.

### Comparative Examples 2 to 5

Each composite material and each beverage were produced in the same manner as in Example 2, except that the phytosterol ester was replaced with a corresponding one of lipid materials described in the following Table 2. The following Table 2 shows the compositions of the beverages and sensory evaluation results thereof.

TABLE 2

| | | | Example | Example | Comparative | Comparative | Comparative | Comparative |
|---|---|---|---|---|---|---|---|---|
| | | | 1 | 2 | Example 2 | Example 3 | Example 4 | Example 5 |
| Compo- | Capsicum oleoresin | | 0.024 Parts | 0.015 Parts | 0.015 Parts | 0.015 Parts | 0.015 Parts | 0.015 Parts |
| sition | Raw | Phytosterol ester | 0.303 Parts | 0.185 Parts | | | | |
| | material | Palm oil | | | 0.185 Parts | | | |
| | lipid | Diglyceride | | | | 0.185 Parts | | |
| | | (“Kenko Econa | | | | | | |
| | | Cooking Oil” | | | | | | |
| | | manufactured by | | | | | | |
| | | Kao Corporation) | | | | | | |
| | | Soybean phospholipid | | | | | 0.185 Parts | |
| | | (“Lecithin” | | | | | | |
| | | manufactured by J- | | | | | | |
| | | OIL MILLS, Inc.) | | | | | | |
| | | Medium-chain fatty | | | | | | 0.185 Parts |
| | | acid triglyceride | | | | | | |
| | | (“COCONARD RK” | | | | | | |
| | | manufactured by | | | | | | |
| | | Kao Corporation) | | | | | | |

| | | | | | | |
|---|---|---|---|---|---|---|
| γ-cyciodextrin | 8.15 Parts | 5.00 Parts | 5.00 Parts | 5.00 Parts | 5.00 Parts | 5.00 Parts |
| Water | 91.413 Parts | 94.69 Parts | 94.69 Parts | 94.69 Parts | 94.69 Parts | 94.69 Parts |
| Citric acid | 0.1 Parts | 0.1 Parts | 0.1 Parts | 0.1 Parts | 0.1 Parts | 0.1 Parts |
| Trisodium citrate | 0.01 Parts | 0.01 Parts | 0.01 Parts | 0.01 Parts | 0.01 Parts | 0.01 Parts |
| Total | 100.00 Parts | 100.00 Parts | 100.00 Parts | 100.00 Parts | 100.00 Parts | 100.00 Parts |

| | | | | | | |
|---|---|---|---|---|---|---|
| Amount of capsaicins contained | 0.001 Parts | 0.006 Parts | 0.006 Parts | 0.006 Parts | 0.006 Parts | 0.006 Parts |
| Sensory evaluation | Almost no | Almost no | Pungency | Pungency | Pungency | Pungency |
| | pungency | pungency | perceived | perceived | perceived | perceived |
| | perceived | perceived | | | | |

### Example 3

### (1) Production of Composite Material

150 parts by weight of a phytosterol ester (San-Ei Gen F. F. I., Inc. “San Sterol No. 3”), and 1.42 parts by weight of a capsicum oleoresin (content of capsaicins: 35.21% by weight) were weighted into a beaker. The mixture was warmed in hot water until the temperature thereof reached 60° C., and the capsicum oleoresin was dissolved. Thus a phytosterol ester in which the capsaicins were dissolved was prepared.

Next, into a measurement kneader (jacket water temperature: 30° C.) of a kneader (manufactured by Brabender, DO-CORDER, DO330 model), 300 parts by weight of γ-cyclodextrin were added. With stirring at 66 rpm, 150 parts by weight of water (at 30° C.) were added thereto. After 5 minutes of stirring, 29 parts by weight of the above-described phytosterol ester containing the capsaicins were added thereto, and the mixture was stirred for 30 minutes. Thus, a composite material was produced.

### (2) Production of Beverage

To 28.82 parts by weight of the composite material, 770.3 parts by weight of water were added to be mixed thereto. Subsequently, 0.8 parts by weight of citric acid and 0.08 parts by weight of trisodium citrate were added to be mixed thereto. This mixture was heated to 93° C. with stirring, and this temperature was kept for 3 minutes to perform heat sterilization. Thereafter, this mixture was filled into a container and cooled. Thus, a beverage in the container was produced. Note that this beverage had a pH of 2.9. The thus obtained beverage had almost no perceptible pungency.

Next, the following Examples 4 to 10 are examples in which a capsicum pepper extract, capsaicin, a ginger extract, a pepper extract, a Japanese pepper extract, an allyl mustard oil or a turmeric extract were used respectively, as the lipophilic component, as shown in Table 3.

### Examples 4 to 10

### (1) Production of Composite Materials

The corresponding one of the lipophilic components described in Table 3 was added to 0.3 parts by weight of a phytosterol ester (San-Ei Gen F. F. I., Inc. “San Sterol Ester NO. 3”). With stirring, the mixture was warmed to 60° C. and the lipophilic component was dissolved. Thus a phytosterol ester in which a pungent component or a bitter component was dissolved was prepared.

Separately, 8.2 parts by weight of γ-cyclodextrin and 4 parts by weight of water (at 75° C.) were mixed with each other, to thereby prepare a mixture (a paste).

To the mixed paste, the phytosterol ester in which the pungent component or the bitter component was dissolved was added. The mixture was kneaded for 10 minutes by using a mortar, while being warmed at 75° C. Thus each composite material was produced.

### (2) Production of Beverages

To each of the composite materials, 87 parts by weight of water were added to be mixed therewith. Subsequently, 0.1 parts by weight of citric acid and 0.01 parts by weight of trisodium citrate were added to be mixed therewith. Next, this mixture was warmed to 93° C. with stirring, and this temperature was kept for 3 minutes to perform heat sterilization. Thereafter, this mixture was filled into a container, and cooled. Thus, each beverage in the container was produced.

### (3) Evaluation of Beverage

As shown in evaluation results in Table 3, the thus produced beverages were superior to those in Comparative Examples shown below in terms of reduction in taste and odor and appearance (floating oil, attachment onto the inner surface of the container).

Next, Comparative Examples 6 to 11 shown below correspond to Examples 4 to 10, except that a palm oil was used in place of the phytosterol ester.

### Comparative Examples 6 to 11

### (1) Production of Composite Materials

The corresponding one of the lipophilic components described in Table 3 was added to 0.3 parts by weight of a palm oil (FUJI OIL CO., LTD. “PALM ACE 10”), which was used in place of the phytosterol ester. With stirring, this mixture was warmed to 60° C., and the lipophilic component was dissolved. Thus a palm oil in which a pungent component or a bitter component was dissolved was prepared.

Separately, 8.2 parts by weight of γ-cyclodextrin and 4 parts by weight of water (at 75° C.) were mixed with each other, to thereby prepare a mixture (a paste).

To the mixed paste, the palm oil in which the pungent component or the bitter component was dissolved was added. The mixture was kneaded for 10 minutes by using a mortar, while being warmed at 75° C. Thus, each composite material was prepared.

### (2) Production of Beverages

To the composite material, 87 parts by weight of water were added to be mixed therewith. Subsequently, 0.1 parts by weight of citric acid and 0.01 parts by weight of trisodium citrate were added to be mixed therewith. Next, this mixture was warmed to 93° C. with stirring, and this temperature was kept for 3 minutes to thereby perform heat sterilization. Thereafter, this mixture was filled into a container and cooled. Thus, each beverage in a container was produced.

### (3) Evaluation of Beverages

Table 3 shows evaluation results of the beverages thus produced in Comparative Examples 6 to 11. Comparative Examples 6 to 11 were obviously inferior to Examples 4 to 10 in terms of appearance (floating oil, deposition onto the inner surface of the container).

Next, the following Comparative Examples 12 to 17 correspond to Examples 4 to 10, except that in stead of y-cyclodextrin, an emulsifier was used.

### Comparative Examples 12 to 17

### (1) Production of Emulsions

The corresponding one of the lipophilic components described in Table 3 was added to 0.3 parts by weight of a phytosterol ester (San-Ei Gen F. F. I., Inc. “San Sterol NO. 3”). With stirring, the mixture was warmed to 60° C., and the lipophilic component was dissolved. Thus, a phytosterol ester in which a pungent component or a bitter component was dissolved was prepared.

Separately, 0.5 parts by weight of an emulsifier (Mitsubishi-Kagaku Foods Corporation, “RYOTO SWA-10D”) were dissolved in 12 parts by weight of water (at 75° C.).

To the solution, the phytosterol ester in which the pungent component or the bitter component was dissolved was added. The mixture was stirred (at 5000 rpm, for 10 minutes) by using a homogenizer. Thus, each emulsion was produced.

### (2) Production of Beverages

To the emulsion, 87 parts by weight of water were added to be mixed therewith. Subsequently, 0.1 parts by weight of citric acid and 0.01 parts by weight of trisodium citrate were added to be mixed therewith. Next, this mixture was warmed to 93° C. with stirring, and this temperature was kept for 3 minutes to perform heat sterilization. Thereafter, this mixture was filled into a container, and cooled. Thus, each beverage in the container was produced.

### (3) Evaluation of Beverages

Table 3 shows the evaluation results of the beverages thus produced in Comparative Examples 12 to 17. Comparative Examples 12 to 17 were obviously inferior to Examples 4 to 10, in terms of reduction in taste and odor, and appearance (floating oil, attachment onto the inner surface of the container).

TABLE 3

| | | | Amount of | | | Evaluation results | | | |
|---|---|---|---|---|---|---|---|---|---|
| | | | component | | | | | Floating oil/ | |
| | | | added (parts | | | | | Attachment | Overall |
| | Lipophilic component | | by weight) | Oil | γCD | Taste | Odor | onto container | evaluation |
| Example 4 | Pungent | Capsicum | 0.000284 | PSE | γCD | Almost no | — | Not | ∘ |
| | component | pepper extract | Parts | | | pungency | | observed | |
| | | (including | | | | perceived | | | |
| Comparative | | capsaicins) | | Palm | γCD | Pungency | — | Observed | x |
| Example 6 | | | | oil | | perceived | | | |
| | | | | | | to some | | | |
| | | | | | | degree | | | |
| Comparative | | | | PSE | None | Apparent | — | Observed | x |
| Example 12 | | | | | (emulsifier) | pungency | | | |
| | | | | | | perceived | | | |
| Example 5 | | Capsaicin | 0.0000947 | PSE | γCD | Almost no | — | Not | ∘ |
| | | (purified product | Parts | | | pungency | | observed | |
| | | (reagent)) | | | | perceived | | | |
| Example 6 | | Ginger extract | 0.026 | PSE | γCD | Almost no | — | Not | ∘ |
| | | (containing | Parts | | | pungency | | observed | |
| | | gingerol and | | | | perceived | | | |
| Comparative | | shogaol) | | Palm | γCD | Almost no | — | Observed | x |
| Example 7 | | | | oil | | pungency | | | |
| | | | | | | perceived | | | |
| Comparative | | | | PSE | None | Apparent | — | Observed | x |
| Example 13 | | | | | (emulsifier) | pungency | | | |
| | | | | | | perceived | | | |
| Example 7 | | Paper extract | 0.0871 | PSE | γCD | Almost no | — | Not | ∘ |
| | | (containing | Parts | | | pungency | | observed | |
| | | piperine) | | | | perceived | | | |
| Comparative | | | | Palm | γCD | Almost no | — | Observed | x |
| Example 8 | | | | oil | | pungency | | | |
| | | | | | | perceived | | | |
| Comparative | | | | PSE | None | Pungency | — | Observed | x |
| Example 14 | | | | | (emulsifier) | perceived | | | |
| | | | | | | to some | | | |
| | | | | | | degree | | | |
| Example 8 | | Japanese | 0.05 Parts | PSE | γCD | Almost no | No | Not | ∘ |
| | | pepper extract | | | | astringent | unpleasant | observed | |
| | | (containing | | | | taste | odor | | |
| | | sanshool) | | | | perceived | perceived | | |
| Comparative | | | | Palm | γCD | Astringent | No | Observed | x |
| Example 9 | | | | oil | | taste | unpleasant | | |
| | | | | | | perceived | odor | | |
| | | | | | | to some | perceived | | |
| | | | | | | degree | | | |
| Comparative | | | | PSE | None | Strong | Unpleasant | Observed | x |
| Example 15 | | | | | (emulsifier) | astringent | sweat-like | | |
| | | | | | | taste | odor was | | |
| | | | | | | perceived | strong | | |
| Example 9 | | Allyl mustard oil | 0.01 Parts | PSE | γCD | Almost no | Almost no | Not | ∘ |
| | | (containing allyl | | | | pungency | irritating | observed | |
| | | isothiocyanate) | | | | perceived | odor | | |
| | | | | | | | perceived | | |
| Comparative | | | | Palm | γCD | Almost no | Almost no | Observed | x |
| Example 10 | | | | oil | | pungency | irritating | | |
| | | | | | | perceived | odor | | |
| | | | | | | | perceived | | |
| Comparative | | | | PSE | None | Pungency | Strong | Observed | x |
| Example 16 | | | | | (emulsifier) | perceived | irritating | | |
| | | | | | | | odor | | |
| | | | | | | | perceived | | |
| Example 10 | Bitter | Turmeric extract | 0.06 Parts | PSE | γCD | Almost no | Almost no | Not | ∘ |
| | component | (containing | | | | bitter taste | earthy odor | observed | |
| | | essential oil | | | | perceived | perceived | | |
| Comparative | | component with | | Palm | γCD | Almost no | Almost no | Observed | x |
| Example 11 | | bitter taste) | | oil | | bitter taste | earthy odor | | |
| | | | | | | perceived | perceived | | |
| Comparative | | | | PSE | None | Strong | Strong | Observed | x |
| Example 17 | | | | | (emulsifier) | bitter taste | earthy odor | | |
| | | | | | | perceived | perceived | | |

* PSE: Phytosterol ester, γCD: γ-cyclodextrin

Next, in the following Examples 11 to 13 composite materials were produced by respective methods different from one another.

### Example 11

### (1) Production of Composite Material

0.003 parts by weight of a capsicum oleoresin (content of capsaicins: 35.2%), which is a pungent component, were added to 0.3 parts by weight of a phytosterol ester (San-Ei Gen F. F. I., Inc., “San Sterol NO. 3”). With stirring, the mixture was warmed to 60° C., and the capsicum oleoresin was dissolved. Thus a phytosterol ester in which the pungent component was dissolved was prepared.

Separately, 8.2 parts by weight of γ-cyclodextrin and 6 parts by weight of water (at 75° C.) were mixed with each other, to thereby prepare a mixture (a paste).

To the mixed paste, the phytosterol ester in which the pungent component was dissolved was added. The mixture was subjected to a homogenizing treatment (at 5000 rpm for 10 minutes). Thus, a composite material was obtained.

### (2) Production of Beverage

To the composite material, 85 parts by weight of water were added to be mixed therewith. Subsequently, 0.1 parts by weight of citric acid and 0.01 parts by weight of trisodium citrate were added to be mixed therewith. Next, this mixture was warmed to 93° C. with stirring, and this temperature was kept for 3 minutes to perform heat sterilization. Thereafter, this mixture was filled into a container, and cooled. Thus, a beverage in the container was produced.

### Example 12

### (1) Production of Composite Material

0.3 parts by weight of a phytosterol ester (San-Ei Gen F. F. I., Inc., “San Sterol NO. 3”) were warmed to 60° C. and dissolved with stirring.

Separately, 8.2 parts by weight of γ-cyclodextrin and

6 parts by weight of water (at 75° C.) were mixed with each other, to thereby prepare a mixture (a paste).

To the mixed paste, the phytosterol ester was added, and the mixture was subjected to a homogenizing treatment (at 5000 rpm for 10 minutes).

To the homogenized product, 0.003 parts by weight of a capsicum oleoresin (content of capsaicins: 35.2%), and 2 parts by weight of water (at 75° C.) were added and the mixture was subjected to a homogenizing treatment (at 5000 rpm for 1 minute). Thus, a composite material was obtained.

### (2) Production of Beverage

To the composite material, 85 parts by weight of water were added to be mixed therewith, and 0.1 parts by weight of citric acid and 0.01 parts by weight of trisodium citrate were added to be mixed therewith. Subsequently, this mixture was warmed to 93° C. with stirring, and this temperature was kept for 3 minutes to perform heat sterilization. Thereafter, the mixture was filled into a container, and cooled. Thus a beverage in the container was produced.

### Example 13

### (1) Production of Composite Material

0.3 parts by weight of a phytosterol ester (San-Ei Gen F. F. I., Inc., “San Sterol NO. 3”) were warmed to 60° C. and dissolved with stirring.

A homogenizing treatment (at 5000 rpm for 10 minutes) was performed on 8.2 parts by weight of γ-cyclodextrin, 0.003 parts by weight of a capsicum oleoresin (content of capsaicins: 35.2%), the above-described phytosterol ester, and 6 parts by weight of water (at 75° C.), to thereby obtain a composite material.

### (2) Production of Beverage

To the composite material, 85 parts by weight of water were added to be mixed therewith, and 0.1 parts by weight of citric acid and 0.01 parts by weight of trisodium citrate were added to be mixed therewith. Subsequently, this mixture was warmed to 93° C. with stirring, and this temperature was kept for 3 minutes to perform heat sterilization. Thereafter, this mixture was filled into a container, and cooled. Thus, a beverage in the container was produced.

### (Evaluation of Beverages)

In all of Examples 11 to 13, a pungency reduction effect was observed. The magnitude of the pungency reduction effect was greatest in Example 11, second greatest in Example 12, and third greatest in Example 13.

Next, in the following Examples 14 and 15 composite materials were produced by using different phytosterol esters. Specifically, in Example 14, “San Sterol NO. 3” of San-Ei Gen F. F. I., Inc. was used, whereas, in Example 15, “phytosterol fatty acid ester” of TAMA BIOCHEMICAL CO., LTD. was used.

### Examples 14 and 15

### (1) Production of Composite Materials

0.003 parts by weight of a capsicum oleoresin (content of capsaicins: 35.2%) were added to 0.3 parts by weight of a phytosterol ester (“San Sterol NO. 3” of San-Ei Gen F. F. I., Inc. (Example 14) or “phytosterol fatty acid ester” of TAMA BIOCHEMICAL CO., LTD. (Example 15)). With stirring, the mixture was warmed to 60° C. and the capsicum oleoresin was dissolved. Thus, a phytosterol ester in which the pungent component was dissolved was prepared.

Separately, 8.2 parts by weight of γ-cyclodextrin and 4 parts by weight of water (at 75° C.) were mixed with each other, to thereby prepare a mixture (a paste).

To the mixed paste, the phytosterol ester in which the pungent component was dissolved was added. The mixture was kneaded for 10 minutes by using a mortar, while being warmed at 75° C. Thus, each composite material was produced.

### (2) Production of Beverages

To the composite material, 87 parts by weight of water were added to be mixed therewith. Subsequently, 0.1 parts by weight of citric acid and 0.01 parts by weight of trisodium citrate were added to be mixed therewith. Next, this mixture was warmed to 93° C. with stirring, and this temperature was kept for 3 minutes to perform heat sterilization. Thereafter, this mixture was filled into a container, and cooled. Thus, each beverage in the container was produced.

### (Evaluation of Beverages)

The beverages of Examples 14 and 15 had equally good pungency reduction effects, and appearances (no floating oil, and no attachment onto the inner surface of the container).

Next, the following Example 16 is a liquid composition (a beverage) in a container. This liquid composition includes a composite material, water and a thickener, and the composite material is in a water dispersed form.

### Example 16

### (1) Production of Composite Material

0.003 parts by weight of a capsicum oleoresin (content of capsaicins: 35.2%) were added to 0.3 parts by weight of a phytosterol ester (San-Ei Gen F. F. I., Inc., “San Sterol NO. 3”). With stirring, this mixture was warmed to 60° C., and the capsicum oleoresin was dissolved. Thus, a phytosterol ester in which the pungent component was dissolved was prepared.

Separately, 8.2 parts by weight of γ-cyclodextrin and 4 parts by weight of water (at 75° C.) were mixed with each other to prepare a mixture (a paste).

To the mixed paste, the phytosterol ester in which the pungent component was dissolved was added. The mixture was kneaded for 10 minutes by using a mortar, while being warmed at 75° C. Thus, a composite material was produced.

### (2) Production of Beverage

To the composite material, citric acid, trisodium citrate, a fermentation-derived cellulose preparation (San-Ei Gen F. F. I., Inc., “SAN ARTIST PX”), granulated sugar, and water were added at a proportion shown in Table 4, and mixed with stirring. Subsequently, this mixture was warmed to 93° C. with stirring, and this temperature was kept for 3 minutes to perform heat sterilization. Thereafter, this mixture was filled into a container, and cooled. Thus, a beverage in the container was produced.

### (3) Evaluation of Beverage

The beverage in the container caused almost no pungency to be perceived, and caused no floating oil and no attachment onto the inner surface of the container. Moreover, in this beverage, the composite material was not deposited, but instead was dispersed uniformly. Note that this beverage had a pH of 3.5.

TABLE 4

| | Mixing proportion |
|---|---|
| Components | (% by weight) |
| The above-described composite material | 12.503 |
| Citric acid | 0.50 |
| Trisodium citrate | 0.25 |
| Fermentation-derived cellulose preparation | 0.60 |
| (San-Ei Gen F.F.I., Inc., “SAN ARTIST PX”) | |
| Granulated sugar | 10.0 |
| Water | 76.147 |
| Total | 100 |

1. A composite material comprising:
a lipophilic component having an irritating taste and/or odor;
a phytosterol ester; and
a cyclodextrin.

2. The composite material according to claim 1,
obtained by mixing the lipophilic component, the phytosterol ester and the cyclodextrin with each other in the presence of water.

3. The composite material according to claim 1,
wherein the lipophilic component is a pungent component or a bitter component.

4. A composition comprising the composite material according to claim 1 blended therein.

5. A liquid composition comprising:
the composite material according to claim 1;
water; and
a thickener,
wherein the composite material is in a water dispersed form.

6. A method for producing a composite material comprising: a lipophilic component having an irritating taste and/or odor; a phytosterol ester; and a cyclodextrin,
the method comprising the step of mixing the lipophilic component, the phytosterol ester and the cyclodextrin with each other in the presence of water, to thereby form the composite material.

7. The method for producing composite material according to claim 6, comprising the steps of:
dissolving the lipophilic component in the phytosterol ester;
preparing a mixture containing the cyclodextrin and water; and
mixing the phytosterol ester which dissolves the lipophilic component with the mixture, to thereby form the composite material.
